(19) United States
(12) Patent Application Publication
(10) Pub. No.: US 2018/0206775 A1
(43) Pub. Date: Jul. 26, 2018

(54) MEASURING MEDICATION RESPONSE USING WEARABLES FOR PARKINSON'S DISEASE

(71) Applicant: THE JOHNS HOPKINS UNIVERSITY, Baltimore, MD (US)

(72) Inventors: Suchi Saria, New York, NY (US); Andong Zhan, Cumberland, MD (US)

(21) Appl. No.: 15/877,640

(22) Filed: Jan. 23, 2018

Related U.S. Application Data
(60) Provisional application No. 62/449,299, filed on Jan. 23, 2017.

(51) Int. Cl. A61B 5/00; G16H 50/50; G16H 40/67; G16H 20/10; A61B 5/11

(52) U.S. Cl. CPC A61B 5/4082; G16H 50/50; G16H 40/67; A61B 2562/0219; A61B 5/112; A61B 5/7267; A61B 5/6898; G16H 20/10

(57) ABSTRACT

An embodiment in accordance with the present invention includes a smartphone based platform that can be used to objectively and remotely measure aspects related to PD (e.g., voice, balance, dexterity, gait, and reaction time), activities of daily living, and PD medicine response. The present invention includes a unified PD-specific remote monitoring platform that incorporates both active and passive tests to provide high frequency monitoring of symptoms and activities of daily living related to PD and medicine response. The platform of the present invention does not require specialized medical hardware.

### CROSS REFERENCE TO RELATED APPLICATIONS

This application claims the benefit of U.S. Provisional Patent Application No. 62/449,299, filed Jan. 23, 2017, which is incorporated by reference herein, in its entirety.

### FIELD OF THE INVENTION

The present invention relates generally to medical informatics. More particularly, the present invention relates to measuring medication response using wearables for Parkinson's disease.

### BACKGROUND OF THE INVENTION

Parkinson disease (PD) is a progressive neurodegenerative disease associated with substantial morbidity, increased mortality, and particularly high economic burden. The prevalence of PD is increasing with as many as one million Americans and an estimated seven to ten million people worldwide living with PD. Direct and indirect costs of PDs are estimated to be nearly $25 billion annually in the United States alone, and are expected to grow significantly as the number of affected individuals increases.

Currently, individuals living with PD have access to care or participate in research primarily during in-person clinic or research visits, which take place at most once every few months. More frequent clinic visits are limited by travel distance, increasing disability, and uneven distribution of doctors. During clinic visits, clinicians assess current disease status and adjust medication for their patients based on response and side effects. However, a key challenge for PD treatment is that PD progression varies among individuals. Furthermore, individuals may exhibit large variations. More specifically, symptoms can fluctuate substantially over the medium term, and the progression is not smooth for everyone.

Accordingly, it is difficult for clinicians to provide optimal treatment for their patients based on these periodic “snapshots” of the disease progression. Therefore, assessments based on clinic visits alone are insufficient, and high frequency remote monitoring is needed to improve the quantity and quality of care for PD. For example, real-time, objective monitoring of daily fluctuations in symptoms can enable timely assessments of disease and response to treatment. These data can enable more subtle adjustments of medications or other therapies for PD and assessment of the efficacy of novel interventions. Existing studies have required the use of specialized and expensive medical devices such as wearable accelerometers and gyroscopes, EEG and passive infrared sensors. Many of these studies have also only reported data collected in the laboratory setting, which does not faithfully represent the patterns of variability that individuals with PD may experience at home. In addition, the majority of past studies have focused on monitoring only one aspect of PD such as dyskinesia, gait, voice, postural sway, and resting tremor. However, PD is a multi-faceted disease with many varied symptoms. Thus, a multi-dimensional approach is needed to continuously monitor all PD symptoms at home.

Mobile phone based tracking and measurement tools offer a promising new avenue for monitoring progressive conditions outside the clinic. The smartphone is becoming one of the most basic necessities. From a recent survey of 170,000 adult Internet users across 32 markets, 80% now own a smartphone. Moreover, without the need for expensive specialized medical hardware, new software tools can be easily downloaded and installed on an individual's smartphone for in-home monitoring.

The most widely-studied and understood symptoms in PD pertain to impairments in the motor subsystem of the central nervous system, including tremor at rest, bradykinesia, rigidity, and postural instability. Other non-motor aspects of the disease include depression, anxiety, autonomic dysfunction, and dementia, which are common and significantly affect health-related quality of life of both individuals with PD and their caregivers.

The rapid rise of wearable consumer devices and smartphone technologies and the need for high frequency monitoring of PD symptoms have led to a proliferation of remote monitoring studies in PD. However, existing studies suffer from the following shortcomings. First, the majority of them rely on specialized medical hardware. For example, studies have used wearable accelerometers and gyroscopes, EEG, treadmill and video camera, or passive infrared sensors. Often these studies require many sensors to be mounted at various positions on the body. Additionally, these commercial medical devices are extremely expensive (often >$3,000 per device excluding software) in comparison to the essential embedded sensing hardware (e.g. MEMS accelerometer, $5) and require the use of proprietary analysis algorithms whose internal operation is not available to scientific scrutiny and independent replication. These requirements significantly limit the use of this technology in the home and community setting, and for large-scale studies of PD symptoms. Secondly, existing studies have typically focused on monitoring only one or two aspects (e.g., dyskinesia, gait, voice, postural sway, tremor, and bradykinesia) of PD. Since in PD no single symptom gives a full picture of an individual's disease state, a multi-dimensional approach is needed to monitor PD comprehensively. Finally, previous studies have primarily reported data from monitoring individuals with PD in the clinical laboratory setting, which are therefore geographically and temporally restricted.

Currently, there is no cure for PD but treatment can help to control the symptoms. For instance, anti-parkinsonian medicines, like levodopa, can help control motor symptoms by increasing dopamine in brain. For individuals with PD who have good medication response, the symptoms of PD can be substantially controlled. By contrast, those in the advanced stages of PD may suffer periods of “wearing off”, i.e. the medication ceases to have any effect, and instead may develop troublesome side effects such as levodopa-induced dyskinesias and problems with impulse control. Because medication response and side effects vary substantially by individual, a personalized medication regime is crucial to maintain quality of life. However, it is difficult for clinicians to determine the optimal regime based on brief moments of observation during clinic visits. Monitoring medication response remotely and objectively is one crucial idea for producing individually optimized PD medication regimes.

Accordingly, there is a need in the art for a non-invasive automated approach to measuring patient mobility and care processes due to the advent of inexpensive sensing hardware and low-cost data storage, and the maturation of machine learning and computer vision algorithms for analysis.

### SUMMARY OF THE INVENTION

The foregoing needs are met, to a great extent, by the present invention which provides a method for Parkinson's disease (PD) monitoring and intervention for a patient including collecting passive and active data related to the patient, wherein active data includes prompting tests of gait, voice, and posture. The method also includes analyzing the passive and active data. The method includes transforming the passive and active data into visual representations of the data for a health care provider. Additionally, the method includes providing updates and reminders to the patient.

In accordance with another aspect of the present invention, passive data further includes data from accelerometers, inertial sensors, GPS, WiFi, and phone usage. The patient is prompted to perform active data testing. The patient can also be prompted to take medicine. A smartphone is provided for collection of the active and passive data. The method includes transmitting the visual representation of the data to the healthcare provider. The method includes prompting the patient to participate in assessments of gait, voice, screen tapping, and posture. The method includes transmitting advice from the health care provider to the patient. The method includes adjusting patient medication dosage based on the passive and active data. Additionally, the method includes analyzing the passive and active data with a rank-based machine learning algorithm.

In accordance with an aspect of the present invention, a system for Parkinson's disease (PD) monitoring and intervention for a patient includes a smart device having sensors. The system includes a processor configured to execute a non-transitory computer readable medium, wherein the non-transitory computer readable medium is programmed for collecting passive and active data related to the patient using a smartphone with an application. Active data includes the application prompting tests of gait, voice, screen tapping, and posture. Passive data includes information collected by the application via features of the smartphone in the background of operation of the smartphone. The non-transitory computer readable medium is also programmed for analyzing the passive and active data. The non-transitory computer readable medium is also programmed for transforming the passive and active data into visual representations of the data for a health care provide and providing updates and reminders to the patient.

In accordance with another aspect of the present invention, the sensors take the form of accelerometers, inertial sensors, GPS, WiFi, and phone usage. The non-transitory computer readable medium is programmed for prompting the patient to perform active data testing. The non-transitory computer readable medium is programmed for prompting the patient to take medicine. The system further includes a smartphone for collection of the active and passive data. The non-transitory computer readable medium further includes transmitting the visual representation of the data to the healthcare provider. The non-transitory computer readable medium further includes prompting the patient to participate in assessments of gait, voice, screen tapping, and posture. The non-transitory computer readable medium further includes transmitting advice from the health care provider to the patient. The non-transitory computer readable medium further includes adjusting patient medication dosage based on the passive and active data. The non-transitory computer readable medium further includes analyzing the passive and active data with a rank-based machine learning algorithm.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a schematic diagram of a method for PD monitoring and intervention, according to an embodiment of the present invention.

FIG. 2 illustrates a schematic diagram further detailing the method for PD monitoring and intervention of the present invention.

FIG. 3 illustrates a map detailing the worldwide participation in the exemplary implementation of the present invention.

FIG. 4 illustrates graphical views of the detailed characteristics of PD participants in an exemplary implementation of the present invention.

FIGS. 5A-5D illustrate graphical views of data collected from both active and passive tests, according to an embodiment of the present invention.

FIGS. 6A and 6B illustrate graphical views of active and passive tests, according to an embodiment of the present invention. FIG. 6A illustrates 185 instances of active tests collected. FIG. 6B illustrates 126 days of passive monitoring, with each line representing one complete passive monitoring session.

FIG. 7 illustrates graphical views of the probability density of feature differences from treatment to baseline among all participants (dashed line at median differences).

FIG. 8 illustrates a graphical view of the relation between accuracy and daily LED.

FIGS. 9A and 9B illustrates graphical views of probability density plots of the feature differences from treatment to baseline from 2 PD participants (dashed lines show median differences).

FIGS. 10A-10C illustrate an internet based front end for an application or program for use on a smartphone or other device, according to an embodiment of the present invention.

FIGS. 11A-11C illustrate graphical views of user monitoring, voice view, and a partial day view, respectively.

FIG. 12 illustrates projections of x₁, x₂, and x₃ on vectors w₁ and w₂ representing two candidate ranking functions.

FIG. 13 illustrates image views of a gait test, tapping test, and voice test according to an embodiment of the present invention.

FIG. 14 illustrates graphical views of correlation of mobile Parkinson Disease Score (mPDS) with traditional Parkinson disease rating scales.

FIGS. 15A-15C illustrate graphical views of sample longitudinal assessments of individuals over six months using the mPDS and the MDS-UPDRS Part III motor score.

FIGS. 16A-16C illustrate graphical views of evaluations of change in mPDS in response to dopaminergic therapy.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention includes a smartphone based platform that is used to objectively and remotely measure aspects related to PD (e.g., voice, balance, dexterity, gait, and reaction time), activities of daily living, and PD medicine response. The present invention includes a unified PD-specific remote monitoring platform that incorporates both active and passive tests to provide high frequency monitoring of symptoms and activities of daily living related to PD and medicine response. The platform of the present invention does not require specialized medical hardware.

The present invention allows the monitoring of PD symptoms remotely by using an application installed on the users' own smartphone. The most significant benefit of this mobile-based approach is high accessibility. In particular, it is inexpensive as no additional purchase is needed for smartphone owners and smartphone-based tests can be conducted anywhere and at any time. This enables assessments at high frequency and large scale in terms of numbers of recruits. Furthermore, it can serve as a potential platform for incorporating care, e.g., telemedicine. Therefore, the present invention can be seen as a preliminary step towards enabling a complete, “closed-loop” remote monitoring and remote intervention tool for PD.

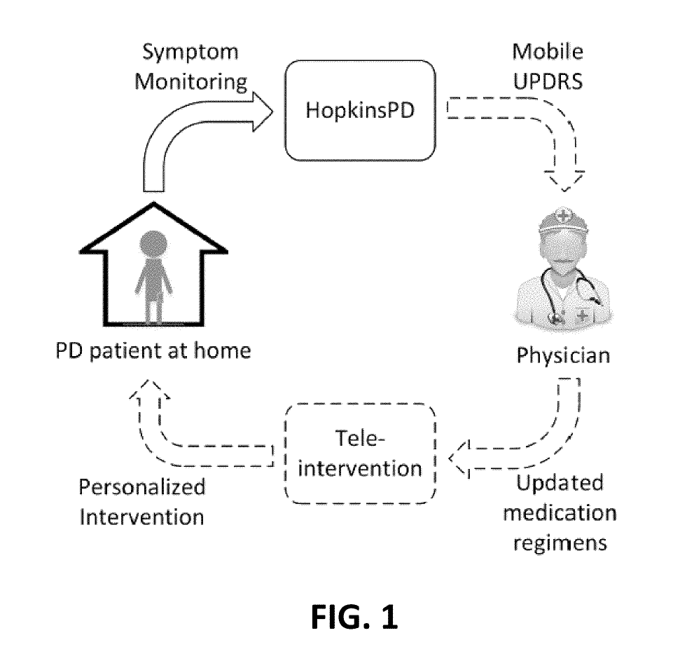

FIG. 1 illustrates a schematic diagram of a method for PD monitoring and intervention, according to an embodiment of the present invention. As illustrated in FIG. 1, the PD patient is able to use the present invention, referred to in the figure as “HopkinsPD,” at home or other locations for monitoring and intervention. The patient uses a monitoring device, such as a smartphone or other similar internet or cellular data transmission enabled device. The monitoring device prompts the patient for data and engages the patient in a number of tests. Exemplary tests are detailed in Table 1, below. The method of the present invention includes uploading the data from the smartphone to a server configured to process the data, especially with respect to the UPDRS. The patient's physician or health care professional then receives the data and can provide further input into the system with respect to treatment for the patient. The method and system of the present invention can also be configured to provide treatment suggestions, guidelines, and protocols based on the UPDRS and other sources. Recommendations can then be forwarded to the patient via the system of the present invention. The system can also be configured to provide the patient with prompts to take medicines or other care related actions in order to improve results of future testing done by the system and method of the present invention. If results are unexpected, the patient can be prompted to confirm that medication has been taken and/or reminded to take medication.

TABLE I
Description of active tests in the present invention

| | | Relevant PD | Related MDS-UPDRS |
|---|---|---|---|
| Test | Description provided to participants | symptoms | III Motor exam |
| Voice | Place the phone to your ear as if making a normal phone call, take a | Dysphonia | 3.1 Speech |
| | deep breath, and say “aaah” for as long and as steadily as you can. | | |
| Balance | Stand up straight unaided and place the phone in your pocket for 30 | Postural instability | 3.12 Postural stability |
| | seconds. | | 3.13 Posture |
| Gait | Stand up and place the phone in your pocket. When the buzzer vibrates | Bradykinesia | 3.8 Leg agility |
| | walk forward for 20 yards; then turn around and walk back. | Freezing of gait | 3.10 Gait |
| | | | 3.11 Freezing of gait |
| Dexterity | Place the phone on a surface such as a desk or table. Tap the buttons | Bradykinesia | 3.4 Finger tapping |
| | alternately with the index and middle finger of one hand, keeping a | Reduced dexterity | 3.5 Hand movements |
| | regular rhythm. | | |
| Reaction | Keep the phone on a surface as before. Press and hold the on-screen | Bradykinesia | 3.5 Hand movements |
| | button (i.e., at the bottom of the screen) as soon as it appears; release | Reduced dexterity | |
| | it as soon as it disappears. | | |
| Rest Tremor* | Sit upright, hold the phone in the hand most affected by your tremor, | Resting tremor | 3.17 Rest tremor amplitude |
| | and rest it lightly in your lap. | | |
| Postural | Sit upright and hold the phone in the hand most affected by your | Postural Tremor | 3.15 Postural tremor of |
| Tremor* | tremor outstretched straight in front of you. | | the hands |

*Tests have been implemented but not used in this study.

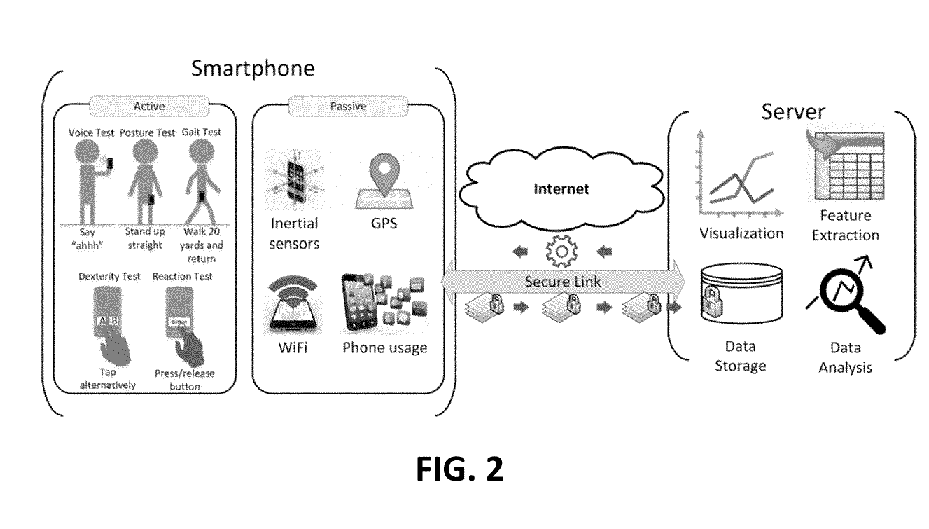

The present invention enables fully-automated capture, compression, encryption, and upload to secure server storage by both actively interacting with smartphone users and passively sensing their daily activities. FIG. 2 illustrates a schematic diagram further detailing the method for PD monitoring and intervention of the present invention. The present invention includes both active and passive testing of the patient's PD symptoms. With respect to the active testing, the patient is prompted to engage in various tests to gauge voice, posture, and gait, for example. Passive testing is done by elements of the monitoring device, such as the inertial sensors, GPS, WiFi, and patient device usage. After the data from the active and passive testing is transmitted to the server, it is processed and visualizations of the data are generated for use by the health care professionals in charge of the patient's care. The core of the mobile application of the present invention is a set of tests to monitor and assess symptoms appearing on the UPDRS scale through smartphones, which consists of:

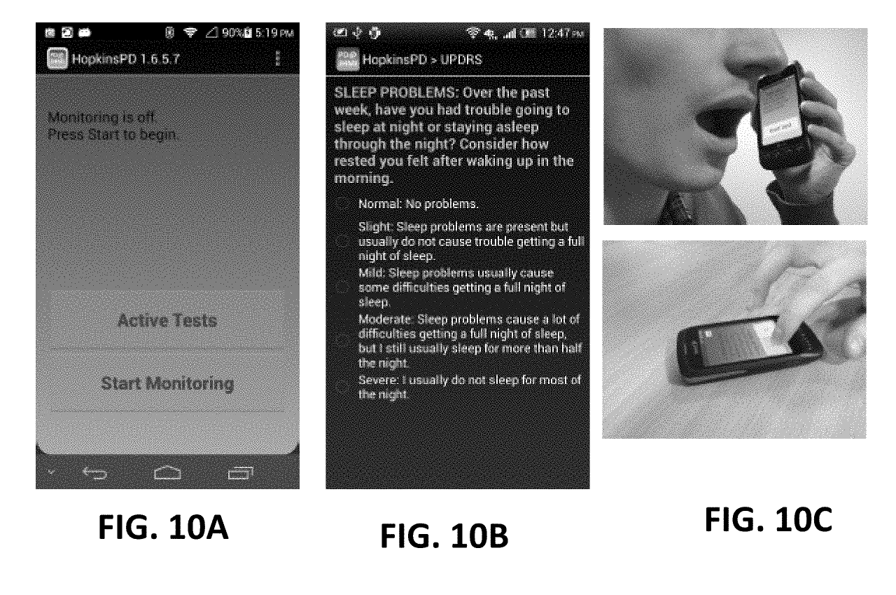

1) Active tests, tests that are initiated and self-administered by the participants at various times during the day: these tests are designed to measure several aspects of motor function such as gait, voice, dexterity, reaction time, and postural instability (balance), using built-in smartphone sensors (See more details in Table I, and visual depiction in FIGS. 10A-10C);

2) Passive tests, running continuously and unobtrusively in the background, are designed to measure aspects of daily living: these use the sensors such as accelerometer, gyroscope, magnetic field strength, GPS location, WiFi parameters, and phone usage logs to measure movement (e.g. whether the individual is experiencing frozen gait or dyskinesia), as well as location and social behavior (e.g. whether they are primarily home-bound or have an active lifestyle). Passive monitoring provides a way to be monitored objectively without interrupting routine activities. The successful monitoring of these daily details may allow comprehensive insight into the behavior and lifestyles of individuals living with PD, which have not been fully investigated in previous studies.

3) Self-reported evaluation of their overall health, mood, and well-being: the significant advantage of such mobile-based questionnaires is that they can be completed outside the clinic. For example, considering that about half to two-thirds of people with PD report that they have memory problems, an on-demand survey system which can probe such problems may be much more accurate and effective than the current approach, in which questionnaires can only be completed during clinic visits thereby relying on the memory of individuals to accurately report their own symptoms over the last few months.

In addition to data collection, the present invention provides HIPAA compliant data streaming to a secure server and web-based analysis and visualization of the resulting data. Additional specifics of the system of the present invention are summarized in the Supplemental Section. In an exemplary implementation of the present invention, participants were identified and recruited using an email database from the Parkinson's Voice Initiative, online media, and patient registries such as the Michael J. Fox Foundations Fox Trial Finder. They were required to understand English and own an Android smartphone with Internet access (e.g. WiFi). After enrollment, participants received a confirmation email with an installation URL to click, which automatically installed the application directly onto their smartphone.

During the exemplary implementation, participants were asked to conduct active tests and passive monitoring daily (self-report surveys were not included in this implementation). Each time the application required the user to perform five active tests measuring voice, balance, gait, dexterity, and reaction time sequentially. These five tests taken by the individual during a single session are referred to as an instance of active tests. The participants were asked to perform two instances of these active tests each day: the first one in the morning just before taking medications, and the second approximately one hour after the first. For healthy controls, they were asked to perform the first in the morning and the second one hour later. It is also possible that the patient be prompted to participate in testing based on passive data collected by the system and method of the present invention.

1) Feature Extraction: Table II provides an exhaustive list of the features extracted from the five types of active tests, along with a brief description of each feature. Acceleration features were based on definitions used in previous studies. Acceleration features were computed from the tri-axial acceleration time series (x, y, and z-axis), as well as the spherical transformation of the tri-axial acceleration time series (i.e., radial distance, polar angle, and azimuth angle). The acceleration features in Table II are applied for these six axes respectively. As the acceleration time series were sampled at irregular time intervals, the Lomb-Scargle periodogram was applied to extract frequency-based features e.g. the dominant frequency component in Hz and its amplitude. All the acceleration features are used by both the balance and gait tests. To extract the voice features, the 20-second audio sample is first divided into 40 frames leading to 0.5 second frame duration. Then, each frame is tagged as containing a ‘voiced’ signal if that frame has amplitude greater than the first quartile of the amplitudes among all frames. Then, for further analysis, the longest consecutive run of voiced frames is selected. The length of the largest consecutive run of voiced frames is the “voice duration” feature. Other features extracted from these voiced frames include dominant frequency and amplitude (Table II). Dexterity features are extracted from the stay duration, that is, the length of time the finger stays touching the screen, and the move duration which is the interval of time between a finger release and the next finger press. The reaction features focus on the lag times of finger reactions (i.e. the time intervals between the stimulus appearing and the finger touch event).

2) Classification: the active tests can be used to detect dopaminergic medication response. A random forest classifier is used to generate a mapping from an active test instance to a discrimination of whether the instance represents off treatment (tests performed before medication) or on treatment (tests performed after medication has been taken). The random forest classifier is an ensemble learning method for classification, regression and other machine learning tasks. This method fits many decision tree classifiers to randomly selected subsets of features and averages the predictions from each of these classifiers. A random forest classifier with 500 trees is used and the splitting criterion is based on Gini impurity. Gini impurity is a standard measure used in classification and regression trees (CART) to indicate the diversity a set of training targets. It reaches its minimum (zero) when all training cases in the node fall into a single target class.

TABLE II
Brief description of features extracted for active

| Feature | Brief Description |
|---|---|
| Acceleration | mean: Mean |
| Features^{a} | std: Standard deviation |
| | Q1: 25^{th} percentile |
| | Q3: 75^{th} percentile |
| | IQR: Inter-quartile range (IQR) (Q3-Q1) |
| | median: Median |
| | mode: Mode (the most frequent value) |
| | range: Data range (max-min) |
| | skew: Skewness |
| | kurt: Kurtosis |
| | MSE: Mean squared energy |
| | En: Entropy |
| | MCR: Mean cross rate |
| | DFC: Dominant frequency component |
| | AMP: Amplitude of DFC |
| | meanTKEO: Instantaneous changes in energy due to |
| | body motion^{b} |
| | ARI: Autoregression coefficient at time tag 1 |
| | DPA: Detrended fluctuation analysis [30] |
| | XCORR: Cross-correlation between two axes |
| | MI: Mutural information between two axes |
| | xEn: Cross-entropy between two axes |
| Voice | Len: Voice duration in seconds |
| Features | AMP: Voice amplitude |
| | F0: Dominant voice frequency |
| | AMP and F0 features include mean, standard deviation, |
| | DFA, and the coefficients of polynomial curve |
| | fitting with degree one and two respectively |
| Dexterity | apply the same feature set (includes mean, standard |
| Features | deviation, Q1, Q3, IQR, median, mode, range, skew, |
| | kurt, MSE, En, meanTKEO, AR1, DFA) on two |
| | groups of tapping intervals: |
| | STAY: length of time finger stays touching the phone |
| | screen |
| | MOVE: time interval between release of touch to the |
| | next touch event |
| Reaction | apply the same feature set on the tags of finger |
| Features | reactions (i.e. the time intervals between the stimulus |
| | appearing and the finger touch event), including sum, |
| | mean, standard deviation. Q1, Q3, IQR, median, |
| | mode, range, skew, kurt, MSE, En, meanTKEO, DFA |

^{a}Acceleration features are used for both balance and gait tests
^{b}TKEO stands for Teager-Kaiser energy operator [31]

Random forests can also be used to assess the relative importance of features in a classification problem. In the case of the present invention, the importance of features reflects how strongly predictive they are of the effect of medication. In addition, to compare against a “null” classifier, a naive benchmark is used—the random classifier. In theory this random classifier should achieve exactly 50% performance accuracy.

A 10-fold cross validation (CV) with 100 repetitions is used to estimate the out-of-sample generalization performance. For each run of 10-fold CV, the original dataset is randomly partitioned into 10 equal-sized subsets. Of the 10 subsets, a single subset is retained as the validation subset (10% of the instances), and the remaining nine subsets used as the training data (90% of the instances). The CV process is then repeated 10 times so that each of the 10 subsets is used exactly once as the validation set. This 10-fold CV process is executed 100 times after permuting the original instance dataset uniformly at random. This provides a distribution of classification accuracies which allows estimation of the mean and standard deviation of the performance of the classifier for unseen datasets, hence controlling for overfitting of the classifier to the single available dataset. Classification results are discussed in detail herein, below. A random classifier predicts the unknown active test group by guesses based solely on the proportion of classes in the dataset, in this case, 50% chance to be a baseline or a treatment instance.

TABLE III
Baseline characteristics

| | PD (N = 121) | Control (N = 105) |
|---|---|---|
| Characteristic | N (%) or mean (SD) | N (%) or mean (SD) |
| Demographic information | | |
| Gender (% male) | 71 (59) | 56 (53) |
| Age (years) | 57.6 (9.1) | 45.5 (15.5) |
| Race (% white) | 104 (86) | 86 (82) |
| College graduate | 100 (83) | 76 (72) |
| Previous participant in PD | 56 (46) | 11 (10) |
| study? (% yes) | | |
| Technology information | | |
| Duration of smartphone | 111 (92) | 97 (93) |
| ownership (>1 year) | | |
| Downloaded other apps | 109 (90) | 98 (94) |
| previously ? (% yes) | | |
| Search for health information | 111 (92) | 98 (94) |
| using plane? (% yes) | | |
| Clinical information | | |
| Care from PD specialist (% | 68 (56) | N/A |
| yes) | | |
| Years since symptoms began | 5 (19.8) | N/A |
| Years since diagnosis | 5 (4.7) | N/A |
| Years on medication(s) | 5 (4.7) | N/A |

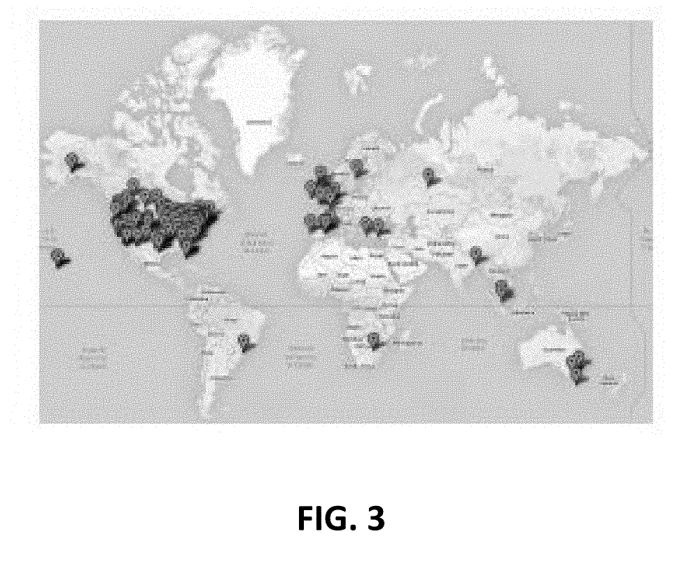

Table III summarizes the characteristics of participants living with PD versus healthy controls. In the present exemplary implementation, 226 individuals (121 PD and 105 controls) contributed data via the present invention. As shown in FIG. 3, these participants come from many of the world's major population centers. FIG. 3 illustrates a map detailing the worldwide participation in the exemplary implementation of the present invention. Each dot indicates an active participant. Meanwhile, a considerable number of healthy controls were also enrolled as participants. The demographics of PD participants and healthy controls are similar, which is important because it will allow for the discovery of PD-specific distinctions between them in future.

Moreover, the familiarity with smartphone usage among the PD participants is comparable to the healthy controls, which suggests that no special requirements are needed in PD-specific remote monitoring. This indicates that smartphone-based remote monitoring approaches which are feasible for the healthy population would also be feasible for PD research.

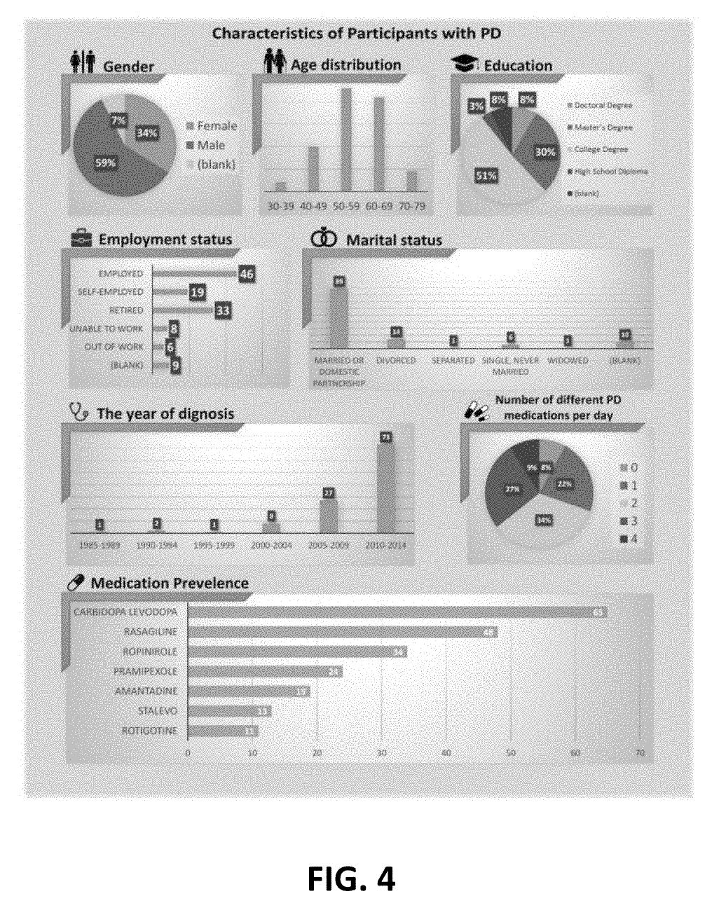

FIG. 4 illustrates graphical views of the detailed characteristics of PD participants in an exemplary implementation of the present invention. The ages of PD participants range from the 30s to 70s, including young onset PD. Participants have varied education, employment and marital status as well. These participants are also in different stages of the disease, diagnosed from 1985 to 2014. Based on this, they would be on various medication regimes. For instance, more than ten types of anti-Parkinsonian drugs are used among them; two-thirds of them need to take more than one type of medication daily to manage their disease. This variety among PD participants helps to ensure that the results of any data analysis are as unbiased as possible.

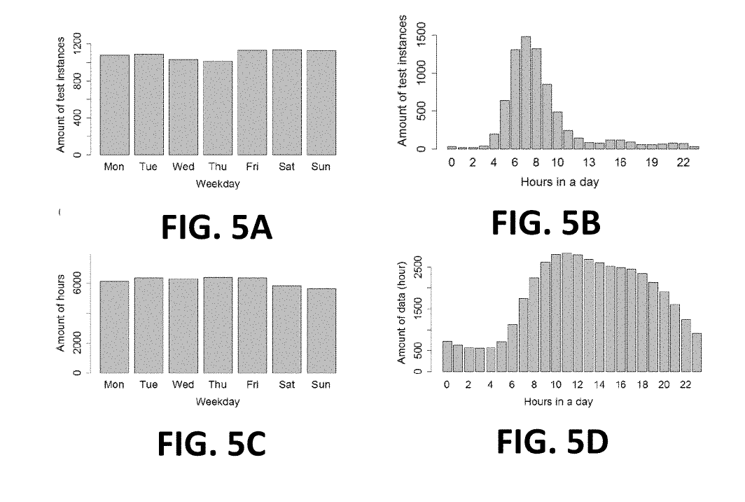

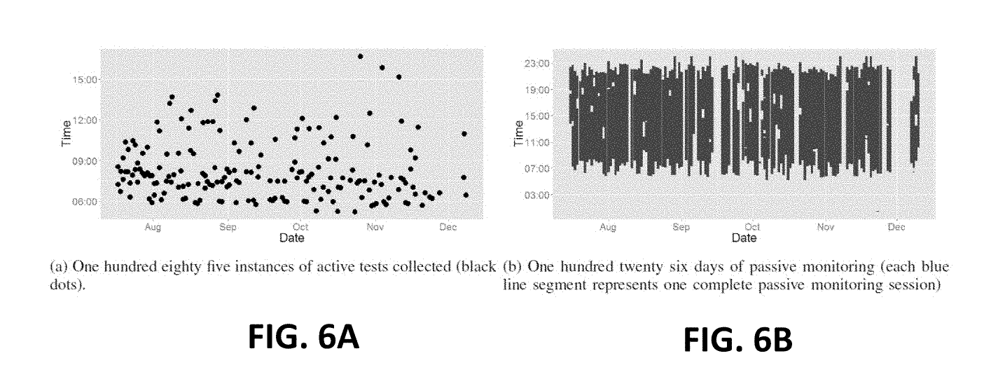

FIGS. 5A-5D illustrate graphical views of data collected from both active and passive tests, according to an embodiment of the present invention. FIGS. 5A and 5C illustrate the number of active test instances and the duration of passive monitoring by day of week respectively, showing weekly data volume collection is effectively uniform. Furthermore, FIGS. 5B and 5D illustrate the number of active test instances and passive data collected by hour of day, respectively. The graphs of FIGS. 5A-5D show most active tests being performed during the morning, and the passive monitoring primarily covering the daytime, particularly from 08:00 to 18:00. As an illustration of high frequency data collection, two timeline charts in FIG. 6B show all active tests and the periods of passive monitoring from a PD participant: this participant started the data collection on Jul. 16, 2014 and recorded 185 instances of active tests and 126 days of passive monitoring. Despite pauses in data collection, which may be attributable to battery depletion, the present invention was still able to collect passive data during most daytime hours. Thus the feasibility of using a smartphone based remote monitoring platform to collect high frequency data for large-scale PD research studies is demonstrated. FIGS. 6A and 6B illustrate graphical views of active and passive tests, according to an embodiment of the present invention. FIG. 6A illustrates 185 instances of active tests collected. FIG. 6B illustrates 126 days of passive monitoring, with each line representing one complete passive monitoring session.

7,653 instances of active tests were collected from both PD participants and healthy controls. To detect medication response for PD participants, all the active test instances from PD participants which can be paired into baseline and treatment are used, giving 4,388 instances in total, to train and evaluate the random forest classifier. To quantify the performance of the random forest classifier in detecting medication response, three commonly used performance measures are calculated: Sensitivity (true positive rate)—proportion of treatment instances correctly identified. Specificity (true negative rate)—proportion of baseline instances correctly identified. Accuracy—proportion of both treatment and baseline instances correctly identified.

From Table IV the accuracy of the random forest classifier is considerably higher than that of random guessing, which indicates that medication response (a) Instances of active tests collected in each day of the week (b) Instances of active tests collected in each hour of the day (c) Hours of passive monitoring in each day of the week (d) Hours of passive monitoring in each hour of the day is detectable by using active tests (p<0:001, two-sided Kolmogorov-Smirnov test). Based on these results, the null hypothesis that random forests have no discriminative power in detecting medication response is rejected.

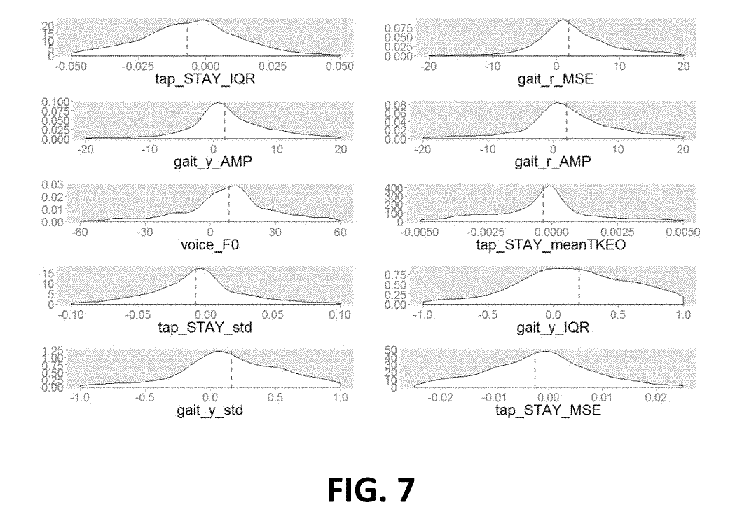

The random forest classifier also indicates the importance of features in creating the classification trees. The ten most important features are described in Table V, and the density plots of the feature differences between baseline and treatment are depicted in FIG. 7. FIG. 7 illustrates graphical views of the probability density of feature differences from treatment to baseline among all participants (dashed line at median differences). The shift of the median differences from zero indicates the improvement of these features after taking medication. Specifically, the following dexterity, voice, and gait features are the most useful in predicting response to dopaminergic medication: Improved tapping rhythm—The decrease of inter-quartile range, standard deviation, mean squared energy and the instantaneous energy changes of the finger pressing intervals suggests that finger tapping movements become faster and more stable after taking medication.

This phenomenon indicates that medication relieves the PD symptoms directly related to hand movements, e.g. bradykinesia. Increased voice pitch—An increase in vocal pitch after medication suggests that medication may relieve specific dysphonias such as monopitch. Improved gait—The increased inter-quartile range, standard deviation, and amplitude of the acceleration signals during walking indicates that PD participants walk more vigorously after taking medication. More specifically, the changes of these features on axis y are more significant than axes x and z. Since the y axis points to the ground during the gait test, it indicates PD participants can lift their feet higher off the floor after taking medication.

TABLE IV

| Method | Sensitivity | Specificity | Accuracy |
|---|---|---|---|
| Random Forest | 69.3 ± 0.5 | 72.7 ± 0.1 | 71.0 ± 0.4 |
| Random Classifier | 49.0 ± 0.1 | 50.9 ± 0.9 | 50.0 ± 0.6 |

Note:
the results are reported in the form average ± standard deviation in percentages (%).
Sensitivity = TP/(TP + TN)
Specificity = TN/(TN + FP)
Accuracy = (TP + TN)/(TP + TN + FP + FN)
Here TP, TN, FP, and FN stand for true positive, true negative, false positive, and false negative, respectively.

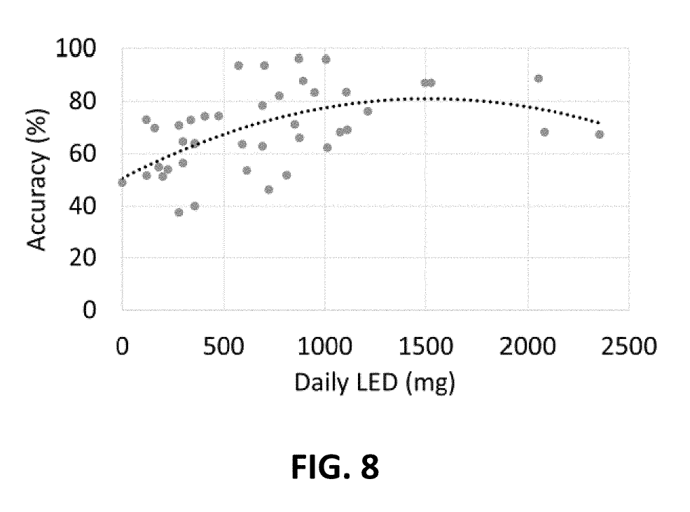

The accuracy of medication response detection varies substantially with total daily levodopa equivalent dose (LED). Here, the daily LED is computed via a standardized formula from daily drug regimes, reported in the pre-study survey. Daily LED provides a useful tool to express dose intensity of different anti-Parkinsonian drug regimes on a single scale, and these regimes do not change within six months. FIG. 8 illustrates a graphical view of the relation between accuracy and daily LED. As shown in FIG. 8, each point indicates the average accuracy of medication response detection for each individual versus the individual's daily LED. The dotted line is the quadratic polynomial regression line. The medication response can be detected more accurately with LED between 500 and 2000 mg than for low dose (less than 500 mg) or high dose (larger than 2000 mg). This is consistent with the fact that individuals with higher dosage of medications are more likely to have (motor) fluctuations in their disease and thus detectable changes in response to treatment.

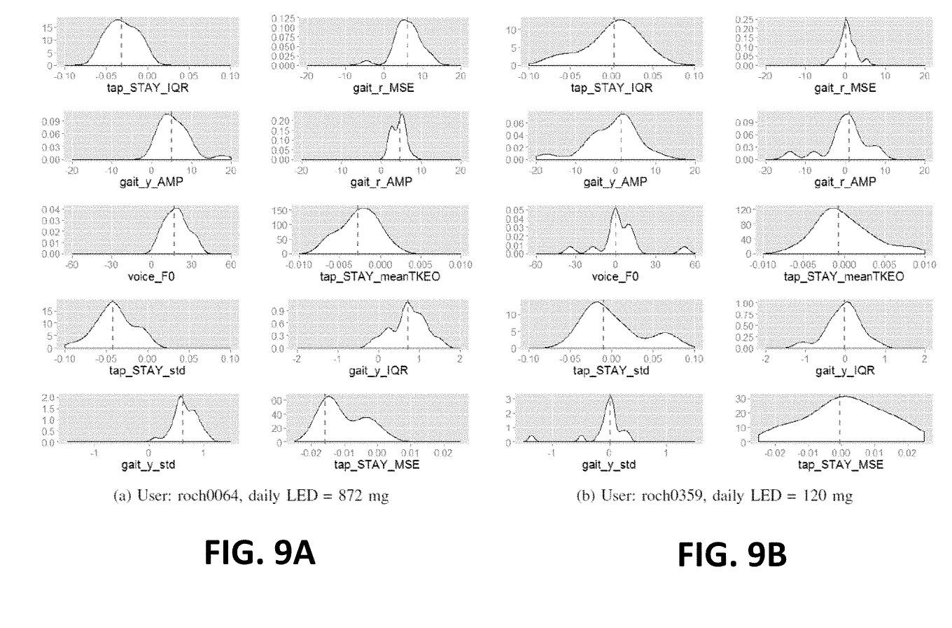

To further understand this, FIGS. 9A and 9B compare the different medication responses between two individuals with different LEDs. FIGS. 9A and 9B illustrate graphical views of probability density plots of the feature differences from treatment to baseline from 2 PD participants (dashed lines show median differences). Participant roch0064, taking a medium LED (872 mg), exhibits a distinct improvement on the ten features, while the improvement is not obvious on roch0359, an individual on low LED (120 mg). For individuals with low LED, the dose is too small to detect a clinically important difference between baseline and treatment; however, for individuals on the largest LED, usually with advanced symptom severity, the medication “wears off” in between doses, but high dosage also induces side effects such as dyskinesias.

TABLE V
The top ten features selected by the random forest classifier

| Feature ID | Test | Description |
|---|---|---|
| tap_STAY_IQR | Dexterity | inter-quartile range of finger |
| | | pressing intervals |
| gait_y_AMP | Gait | the amplitude of the dominant |
| | | frequency on axis y |
| voice_F0 | Voice | the dominant voice frequency |
| tap_STAY_std | Dexterity | standard deviation of finger |
| | | pressing intervals |
| gait_y_std | Gait | standard deviation of axis y |
| gait_r_MSE | Gait | mean squared energy of the radial |
| | | distances |
| gait_r_AMP | Gait | the amplitude of the dominant |
| | | frequency of the radial distances |
| tap_STAY_meanTKEO | Dexterity | mean TKEO of finger pressing |
| | | intervals |
| gait_y_IQR | Gait | inter-quartile range of axis y |
| tap_STAY_MSE | Dexterity | mean squared energy of finger |
| | | pressing intervals |

Compared with consumer wearable devices and specialized medical equipment, smartphones are far more pervasive (wearable devices are used by only less than 10% of online adults, according to a recent survey). Moreover, the smartphone-based platform of the present invention is a more flexible and comprehensive toolkit compared to previous studies based on a single wearable device. The present invention can take the form of an “app” or program for use on a number of different device platforms. The smartphone is the preferable embodiment, as it is a “central hub” for remote monitoring of symptoms in PD. Wearable devices like a wristband or a smart watch could be useful accessories for certain important purposes, e.g. sleep monitoring. It may also be that, in the future, a smart watch with sufficient capability could entirely replace the smartphone. Regardless, it is possible to integrate wearable devices into the present invention. Data streams from future wearable devices would be another kind of passive test, and these data can be synchronized to the phone via Bluetooth and securely uploaded to the server using the existing framework. This would allow raw sensor data collected from wearable devices to be accessed by PD researchers.

Current clinical monitoring of PD is low-frequency and based on periodic clinic visits. In contrast, the present invention is directed to a novel mobile smartphone-based platform to monitor PD symptoms frequently and remotely. By using the controlled tests carried out using only the built-in smartphone sensors, initial experiments using a set of features extracted from the sensor data and random forest classification, is able to detect medication response with 71.0 (0.4)% accuracy. Given the feasibility of using smartphone-based monitoring platform to collect high frequency clinical data and these preliminary results on medication response detection, future studies could devise novel models to track both the fluctuations in symptoms and medication response over time in order to assess disease progression, medication adherence, and, finally, help clinicians make decisions on appropriate therapeutic interventions informed by objective, near real-time, data.

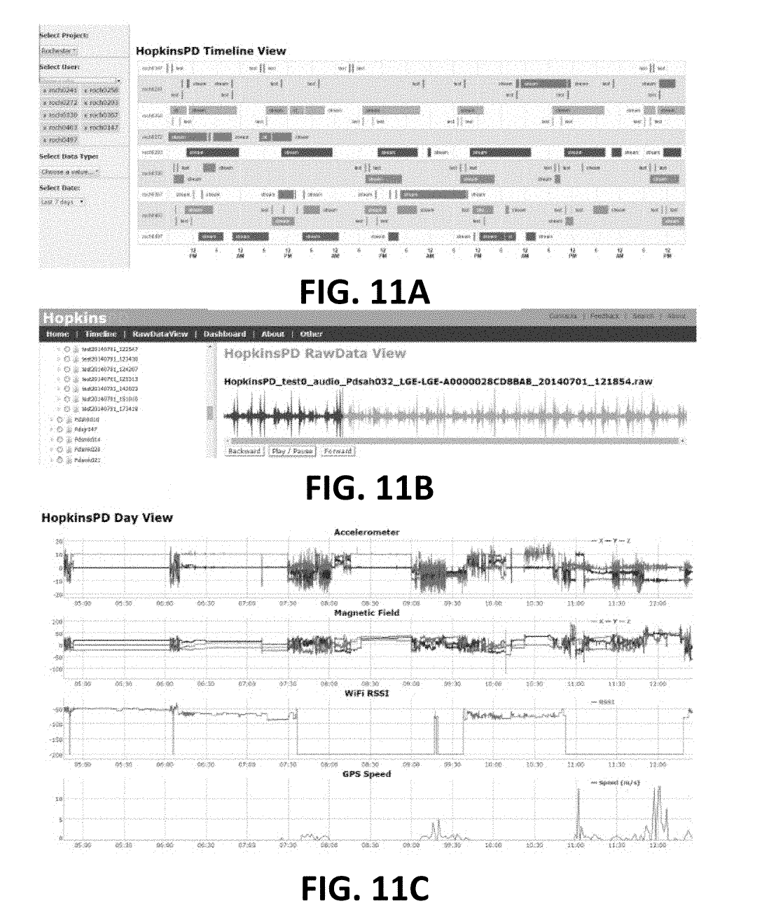

FIGS. 10A-10C illustrate an internet based front end for an application or program for use on a smartphone or other device, according to an embodiment of the present invention. The web front-end, illustrated in FIGS. 10A-10C, is designed to make PD studies manageable for researchers. It provides: Detailed project configuration. The present invention allows researchers to customize the application before launching. In particular, researchers can configure which active tests are enabled, which sensor data to collect during passive monitoring, and which questionnaires to activate. To bring this to light, they can turn on or turn off GPS data collection and configure the sampling frequency, e.g., once per minute or once per hour. The project configuration is scripted using a simple XML, file. A timeline view is provided for researchers to track data collection progress remotely, as (a) Primary interface (FIG. 10A) (b) Self-report (FIG. 10B) (c) Active tests as well as user participation in near real-time (FIG. 10C). An interactive interface is provided in this view so that researchers can specify a range of subjects and time. FIGS. 11A-11C illustrate graphical views of user monitoring, voice view, and a partial day view, respectively.

To explore and visualize the multidimensional data from both active and passive tests, there are two different views provided: a) a test detailed view displaying detailed sensor data plots with zooming. For instance, researchers can visualize the three dimensional acceleration sensor data collected from the accelerometer when the subject is performing a gait test, or play the sound recorded during the voice test; b) a day view summarizing daily passive tests on a dashboard consisting of various charts representing time-series of movement sensor data (e.g., acceleration and compass), location (GPS coordinates on a map), users' interaction with the phone (e.g. app usage and phone calls), and resource consumption (e.g. battery level). This allows researchers to monitor users' daily activities. Examples of data visualization are shown in FIG. 11.

To meet HIPAA requirements, the system must not broadcast identifiable patient data and must guarantee the authenticity of the data it captures. The present invention is designed to be HIPAA-compliant to maintain the integrity of Protected Health Information (PHI) for all participants, which is necessary in that even though personal information such as names are excluded from the platform, PHI could be inferred from data collected from smartphones. Taking GPS as an example, coordinates collected on the phone may expose the participants' home or work address. Therefore, the backend of the present invention has implements the following:

All data is immediately encrypted after collection on the phone; secondly, all data uploaded to the server and all results generated from that data are encrypted and stored on a managed protected server with restricted access. The present invention implements a unified upload manager which uploads data collected via the mobile frontend. This includes HTTPS-based encrypted upload, error handling, and retry mechanisms.

The account management and access control are support authorized individual data access in concurrent deployment settings. A user account can be created by an administrator. Users are authenticated based on their account credentials. The access control in the present invention is designed at a project level so that for an account authorized to access Project A, data collected from participants in Project A alone are accessible to this account. This design isolates projects from one another, thus allowing multiple studies to be securely managed and deployed on the same backend. The above functionalities greatly assist researchers in running remote PD studies and provide a secure data streaming service to protect the data collection process.

A DSS learning algorithm is used in conjunction with the present invention in order to process the data received from the active testing and passive monitoring of the PD patients. The learning algorithm associated with the present invention provides a scalable and automatic approach to learning disease severity scores in new disease domains and populations. The learning algorithm only requires a means for obtaining clinical comparisons—ordered pairs comparing disease severity state at different times. This form of supervision is more natural to elicit than asking clinical experts to map the disease severity score, or encoding an accurate model of disease progression. Moreover, this supervision can often be generated automatically. The present invention allows experts to tune the quality of the score by increasing the granularity and amount of supervision given. The algorithm learns scores that are consistent with clinical expectations. For example, changes in the severity score over consecutive time periods are smooth and the score is higher in periods adjacent to an adverse event. Additionally, the score is sensitive to changes in disease severity state due to therapies.

The algorithm of the present invention takes into account a number of covariates. These include covariates such as age, gender, and clinical history (e.g., presence or absence of a clinical condition such as AIDS or Diabetes) obtained at the time of admission; time-varying measurements such as heart rate, respiratory rate, urine volume obtained throughout the length of stay; and text notes summarizing the patients evolving health status. These data are processed and transformed into tuples <xᵢ^{p},tᵢ^{p}> where xᵢ^{p}ϵ [symbol] ^{d} is a d-dimensional feature vector associated with patient pϵP at time tᵢ^{p} for iϵ{1, . . . , T^{p}} and T^{p} is the total number of tuples for patient p. A feature vector xᵢ^{p} contains raw measurements (e.g., last measured heart rate or last measured white blood cell count) and features derived from one or more measurements (e.g., the mean and variance of the measured heart rate over the last six hours or the total urine output in the last six hours per kilogram of weight). The problem of learning a DSS function is defined by the sets O and S of pairs of tuples from the set D of all tuples, and by the set G of permissible DSS functions. The set O contains pairs of tuples (<xᵢ^{p},tᵢ^{p}>; <xᵢ^{q},tᵢ^{q}>) that are ordered by severity based on clinical assessments. Each of these paired tuples is referred to as a clinical comparison and the set O as the set of all available clinical comparisons. For notational simplicity, xᵢ^{p} corresponds to a more severe state than xⱼ^{q}. These clinical comparisons can be obtained by presenting clinicians with data xᵢ^{p} for patient pϵP at time tᵢ^{p} and data xⱼ^{q} for patient qϵP at time tⱼ^{q}. For each such pair of feature vectors, the clinical expert identifies which of these correspond to a more severe health state; the expert can choose not to provide a comparison for a pair where the severity ordering is ambiguous. These pairs can also be generated in an automated fashion by leveraging existing clinical guidelines.

The set S contains pairs of tuples (<xᵢ^{p},tᵢ^{p}>; <xᵢ^{p}+1,tᵢ^{p}+1>) that correspond to feature vectors that are taken from the same patient p at consecutive time steps tᵢ^{p} and tᵢ^{p}+1. These pairs are used to impose smoothness constrains on the learned severity scores. The pairs in S are referred to as the smoothness pairs. Finally, the set G contains a parameterized family of candidate DSS functions g that map feature vectors x to a scalar severity score. The goal is to identify a function gϵG that quantifies the severity of the disease state represented by a feature vector x. In particular, this function should correctly order any pair (x; x′) of feature vectors by their severity, and the resulting score should be temporally smooth to mimic the natural inertia exhibited by the biological system. Empirical risk minimization is used to identify such a function g. Namely, objective function C^{g} is constructed that maps functions gϵG to their empirical risk. The first of the two terms in C^{g} is

$${\sum }_{( < {x}_{i}^{p} , {t}_{i}^{p} > , < {x}_{i + 1}^{p} , {t}_{i + 1}^{p} > ) ∈ S} {[ \frac{g ( {x}_{i + 1}^{p} ) - g ( {x}_{i}^{p} )}{{t}_{i + 1}^{p} - {t}_{i}^{p}} ]}^{2} . \qquad ( 1 )$$

This term penalizes DSS functions that exhibit large changes in the severity score over short durations, hence encouraging selection of temporally smooth DSS functions. The second term in C^{g} penalizes g for pairs of tuples (<xᵢ^{p},tᵢ^{p}>; <xⱼ^{q},tⱼ^{q}>)ϵ0 for which the severity ordering induced by g on vectors xᵢ^{p} and xⱼ^{q} is inconsistent with the ground truth clinical assessment. i.e., g(xᵢ^{p})<g(xⱼ^{q}).

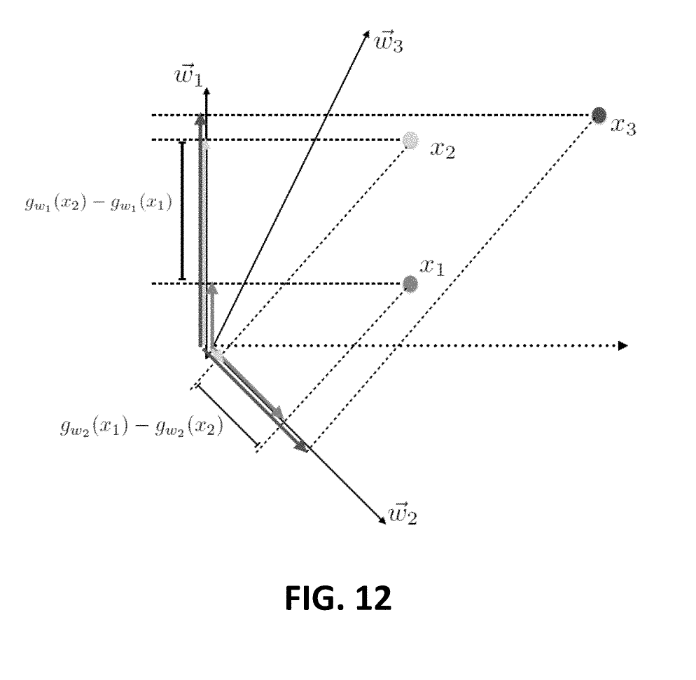

Linear DSS functions, i.e., DSS functions of the form g_{w}(x)=w^{T}x are referred to as L-DSS. Soft max-margin training is used to maximize the distance between the pairs that are at different severity levels while keeping the distance between the consecutive pairs smooth. Consider the toy example shown in FIG. 12. FIG. 12 illustrates projections of x₁, x₂, and x₃ on vectors w₁ and w₂ representing two candidate ranking functions. Ranking is induced by the differences in projections. Let D contain the three feature vectors {x₁; x₂; x₃} where x₁ϵR², and O contain the pairs (x₂; x₁) and (x₃; x₂), i.e., feature vectors x₂ and x₃ have higher disease severity than x₁ and x₂ respectively. Max-margin ranking seeks to find a vector w such that the margin between pairs of different severity levels is maximized. In the example, parameter vectors w₁, w₂ and w₃ for three candidate ranking functions are shown in FIG. 12. For each feature vector x, the assigned (severity) score for a given ranking function parameter w₁ is computed as the projection, g_{wi} (x), of x on wᵢ. The induced ranking between two vectors x₁ and x₂ is computed based on the margin which is defined as the difference in their projections. In the example shown, the rankings induced by both g_{w1} and g_{w3} correctly order all pairs in O, i.e.,

gw₁(x₃)>gw₁(x₂)>gw₁(x₁) and gw₃(x₃)>gw₃(x₂)>gw₃(x₁),

while the rankings induced by w₂ do not. Furthermore, w₃ also induces an ordering with a larger margin between the pairs in O. Margin-maximization leads to an ordering that is more robust with respect to noise in x.

More formally, for each pair of feature vectors (xᵢ, xⱼ)ϵO, the margin of their separation is defined by the function g_{w}(⋅) as=μ_{i,j}^{w}(xᵢ)−g_{w}(xⱼ). The maximum-margin approach suggests that generalization and robustness of the learned separator can be improved by selecting w that maximizes the number of tuples that are ordered correctly (i.e., μ_{i,j}^{w}>0) while simultaneously maximizing the minimal normalized margin μ_{i,j}^{w}∥w∥. Using the standard soft max-margin framework, the SVMRank algorithm approximates the above-mentioned problem as the following convex optimization program:

$${min}_{w , {ζ}_{O}^{i , j}} [ \frac{1}{2} \vert \vert w {\vert \vert}^{2} + \frac{{λ}_{O}}{\vert O \vert} {\sum }_{( {x}_{i} , {x}_{j} ) ∈ O} {ζ}_{O}^{i , j} ] \\ subject to the following ordering contraints \text{:} \\ ∀ ( {x}_{i} , {x}_{j} ) ∈ O \text{:} {g}_{w} ( {x}_{i} ) - {g}_{w} ( {x}_{j} ) \geq 1 - {ζ}_{O}^{i , j} and {ζ}_{O}^{i , j} \geq 0 \qquad ( 2 )$$

For the algorithm for learning linear DSS functions, sets O and S contain feature vectors belong to more than one patients at varying times. The soft-max margin objective with the additional term, shown in Eq. (1), encourages temporal smoothness. The full L-DSS algorithm is:

$${min}_{w , {ζ}_{O}^{i , j}} [ \frac{1}{2} \vert \vert w {\vert \vert}^{2} + \frac{{λ}_{O}}{\vert O \vert} {\sum }_{( < {x}_{i}^{p} , {t}_{i}^{p} > , < {x}_{j}^{q} , {t}_{j}^{q} > ) ∈ O} {ζ}_{O}^{( p , i ) , ( q , j )} + \frac{{λ}_{S}}{\vert S \vert} {\sum }_{( < {x}_{i}^{p} , {t}_{i}^{p} > , < {x}_{i + 1}^{p} , {t}_{i + 1}^{p} > ) ∈ S} {[ \frac{{g}_{w} ( {x}_{i + 1}^{p} ) - {g}_{w} ( {x}_{i}^{p} )}{{t}_{i + 1}^{p} - {t}_{i}^{p}} ]}^{2} ] \\ subject to the following ordering contraints \text{:} \\ ∀ ( < {x}_{i}^{p} , {t}_{i}^{p} > , < {x}_{j}^{q} , {t}_{j}^{q} > ) ∈ O \text{:} {g}_{w} ( {x}_{i}^{p} ) - {g}_{w} ( {x}_{j}^{q} ) \geq 1 - {ζ}_{O}^{( p , i ) , ( q , j )} and \\ ∀ ( < {x}_{i}^{p} , {t}_{i}^{p} > , < {x}_{j}^{q} , {t}_{j}^{q} > ) ∈ O \text{:} {ζ}_{O}^{( p , i ) , ( q , j )} \geq 0 \qquad ( 3 )$$

Here, the coefficients λ o and λ s control the relative degree of emphasis on the smoothness versus the margin-maximization component of the objective. For a given setting of o, different choices of λ s yield trajectories with differing levels of smoothness. An appropriate choice of λ s could be determined by the clinical user based on the rate of change in severity that is to be expected in that domain. For example, in sepsis, changes in severity do not occur within minutes while in many cardiac conditions, rapid changes in severity can occur. Alternately, this parameter can be set using cross-validation to optimize performance for a particular application of DSS.

In Eq. (3), for every value of w, the optimal values of ζ_{O}^{(p,i)(q,j)} are given by

ζ_{O}^{(p,i)(q,j)}=max {0,1−(gw(xᵢ^{p})−gw(xⱼ^{q}))}. (4)

Substituting Eq. (4) and g_{w}(x)=w^{T}x in Eq. (3), the following unconstrained convex optimization formulation is obtained:

$${min}_{w} \frac{1}{2} \vert \vert w {\vert \vert}^{2} + \frac{{λ}_{O}}{\vert O \vert} {\sum }_{( < {x}_{i}^{p} , {t}_{i}^{p} > , < {x}_{j}^{q} , {t}_{j}^{q} > ) ∈ O} max \{ 0 , 1 - {w}^{T} ( {x}_{i}^{p} - {x}_{j}^{q} ) \} + \frac{{λ}_{S}}{\vert S \vert} {\sum }_{( < {x}_{i}^{p} , {t}_{i}^{p} > , < {x}_{i + 1}^{p} , {t}_{i + 1}^{p} > ) ∈ S} {[ \frac{{w}^{T} ( {x}_{i + 1}^{p} - {x}_{i}^{p} )}{{t}_{i + 1}^{p} - {t}_{i}^{p}} ]}^{2} \qquad ( 5 )$$

The primal form of this optimization program is solved as follows. The terms of the form max{0, a}, also called the hinge loss, are not differentiable at α=0. These terms with the Huber loss Lₕ for 0<h<1 are given by

$${L}_{h} ( a ) = \{ \begin{matrix}0 , & if a < - h \\ \frac{{( a + h )}^{2}}{4 h} , & if \vert a \vert \leq h \\ a , & if a > h \end{matrix}$$

This approximation yields the following unconstrained, convex, twice-differentiable optimization problem:

$$\\ \begin{matrix}L \text{-} DSS Objective \text{:} & \begin{matrix}{min}_{w} \frac{1}{2} {‖ w ‖}^{2} + \\ \frac{{λ}_{O}}{\vert O \vert} {\sum }_{( 〈 {x}_{i}^{p} , {t}_{i}^{p} 〉 , 〈 {x}_{j}^{q} , {t}_{j}^{q} 〉 ) ∈ O} {L}_{h} ( 1 - {w}^{T} ( {x}_{i}^{p} - {x}_{j}^{q} ) ) + \\ \frac{{λ}_{S}}{\vert S \vert} {\sum }_{( 〈 {x}_{i}^{p} , {t}_{i}^{p} 〉 , 〈 {x}_{i + 1}^{p} , {t}_{i + 1}^{p} 〉 ) ∈ S} {[ \frac{{w}^{T} ( {x}_{i + 1}^{p} - {x}_{i}^{p} )}{{t}_{i + 1}^{p} - {t}_{i}^{p}} ]}^{2}\end{matrix}\end{matrix} \qquad ( 6 )$$

This optimization program is solved using the Newton-Raphson algorithm. In many disease domains, assuming a linear mapping between the measurements and the latent disease severity may be too restrictive. For example, ranges for measurements values that are considered to be normal (or from a low-severity state) are often age dependent or clinical history dependent. Consider an individual with a pre-existing kidney condition; he or she is likely to have a worse baseline creatinine level (a test that measures kidney function) compared to an individual with fully-functioning kidneys. Thus, when measuring changes in severity related to the kidney, these individuals are likely to manifest a disease differently.

To learn non-linear DSS functions, g is represented as a weighted sum of regression trees. Alternate choices for learning non-linear DSS functions exist including extending the soft-margin formulation presented for learning L-DSS via use of the “kernel-trick”. Here boosted regression trees are extended as this is one of the most widely used algorithms for ranking.

The hypothesis class G includes all linear combinations of shallow regression trees, i.e., functions of the form g(x)=Σₖ₌₁^{K}αₖfₖ(x), where fₖ for k=1, . . . , K are shallow (limited-depth) regression trees and K is finite. In experiments, K is set to 5. Similar to the objective for L-DSS in Eq. (6), the NL-DSS objective is constructed to identify gϵG that maximizes the dual criteria of ordering accuracy and temporal smoothness as:

$$\begin{matrix}NL \text{-} DSS \\ Objective\end{matrix} \text{:} {C}^{g} ( g ) = \frac{1}{\vert O \vert} {\sum }_{( < {x}_{i}^{p} , {t}_{i}^{p} > , < {x}_{j}^{q} , {t}_{j}^{q} > ) ∈ O} {L}_{h} ( 1 - ( g ( {x}_{i}^{p} ) - g ( {x}_{j}^{q} ) ) ) + \frac{{λ}_{S}}{\vert S \vert} {\sum }_{( < {x}_{i}^{p} , {t}_{i}^{p} > , < {x}_{i + 1}^{p} , {t}_{i + 1}^{p} > ) ∈ S} {[ \frac{g ( {x}_{i + 1}^{p} ) - g ( {x}_{i}^{p} )}{{t}_{i + 1}^{p} - {t}_{i}^{p}} ]}^{2} \qquad ( 7 )$$

Note that since the soft max-margin formulation is not defined for a non-linear classifier the term ∥w∥²/2 is dropped. Thus, without loss of generality, λ o can be replaced by 1. Now, the relative emphasis on the smoothing versus the ordering components are changed by varying λ s.

The NL-DSS objective is optimized using the gradient boosted regression trees (GBRT) learning algorithm. Gradient boosting methods grow g incrementally, in a greedy fashion, by adding a weak learner—in this case, a regression tree—at each iteration. A tree that most closely approximates the gradient of C^{g} evaluated at g obtained in the previous iteration is added.

The per-iteration computational complexity of this approach is equivalent to the computational complexity of building a single regression tree, which is |T| log |T|, where |T| is the number of unique tuples in the set O∪S of tuple pairs.

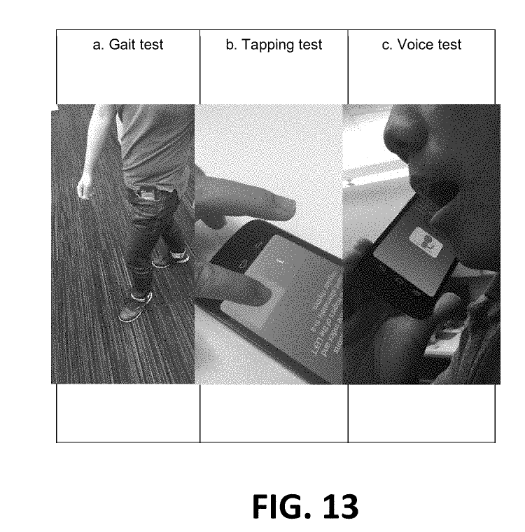

In an exemplary implementation of the present invention, a smartphone-derived severity score for PD is used to provide an objective measurement of symptoms inside and outside of clinical settings. This exemplary implementation is not meant to be considered limiting and is only include as an example of the present invention. Any implementation known to or conceivable to one of skill in the art is also considered within the scope of the present invention. Such data is valuable for clinical care and drug development. In the exemplary implementation, a smartphone application that incorporated tests for voice, finger dexterity, gait, postural instability, and reaction time tests for participants to complete was used. FIG. 13 illustrates image views of a gait test, tapping test, and voice test according to an embodiment of the present invention. Various sensors embedded in the smartphone are used to capture and record these activities. The activities could be completed as often as desired by the participant, including both before and after dopaminergic therapy.

Individuals that participated in the exemplary implementation of the present invention, downloaded the HopkinsPD smartphone application and were asked to regularly complete the smartphone activities alongside traditional in-person clinical assessments, including the MDS-UPDRS Parts III and IV, the Hoehn & Yahr stage, and the Timed Up and Go Test at baseline, month 3, and month 6. The MDS-UPDRS Part I (non-motor experiences of daily living) and Part II (motor experiences of daily living) were emailed to the participants to complete after their in-clinic visits. At month 6, individuals with PD were invited to complete off (>12 hours from last dopaminergic medication) and on (60-90 minutes after dopaminergic medication) assessments in the clinic.

A subset of participants from the first recruitment phase completed one or more pairs of the full set of five activities before and after their first dose of dopaminergic medication each morning over six months. These individuals constituted the development set used for the learning model's parameter estimation and were labeled the active users cohort.

Collected sensor data from HopkinsPD was processed to extract feature vectors for each of the five test activities (e.g. finger tapping speed and inter-tap interval, among others, from the finger tapping activity); a total of 435 unique features were extracted.

A rank-based machine learning algorithm—disease severity score learning (DSSL)—was used to create the mobile Parkinson disease score (mPDS). The algorithm weights the 435 features to produce a severity score. In order to determine each feature's weight in generating the mPDS, DSSL exploits example pairs of times that are rank ordered in severity, assuming that the severity of symptoms at time tᵢ is less than that at time tⱼ. The severity of symptoms immediately preceding medication administration is assumed to be higher than that one hour after medication. Given many such pairs, DSSL estimates a score by optimizing an objective function that seeks to correctly rank as many of the pairs as possible. The mPDS is scaled between 0 and 100, where values close to 0 reflect low motor symptom severity while those closer to 100 reflect high severity.

One general kind of approach for creating a severity score algorithm is based on supervised learning: here, experts evaluate the participants at multiple time points to provide the clinical, “gold-standard” score at each time point (e.g., MD S-UPDRS score). Based on these evaluations, a regression function is estimated that maps features (algorithms such as sensor data variability, complexity and summarized frequency information) derived from the smartphone sensor data collected during the smartphone activities into a continuous or discrete-valued score. The key challenge of using such an approach is that it relies heavily on obtaining a large number of gold-standard clinical evaluations which are very expensive and time-consuming to collect.

Instead, a rank based machine learning algorithm—disease severity score learning (DSSL) is used to create the mobile Parkinson disease score (mPDS). In order to estimate a score from feature data, DSSL uses weak supervision where the resulting labels may have an associated error rate²⁷. For example, to estimate mPDS parameters, DSSL exploits example pairs of times that are rank ordered in severity such that the severity of symptoms at time tᵢ is less than that at time t₁. Using the data collected in this study, such example pairs were easily obtained: for an individual responding to medication, the severity of symptoms at a time right before medication administration is assumed to be higher than that an hour after taking their medications.

Given many such pairs, DSSL estimates a score by optimizing the objective shown in Equation 8 below:

$${min}_{w} \frac{1}{2} \vert \vert w {\vert \vert}^{2} + \frac{{λ}_{O}}{\vert O \vert} {\sum }_{( < {x}_{i}^{p} , {t}_{i}^{p} > , < {x}_{j}^{q} , {t}_{j}^{q} > ) ∈ O} {L}_{h} ( 1 - {w}^{T} ( {x}_{i}^{p} - {x}_{j}^{q} ) ) + \frac{{λ}_{S}}{\vert S \vert} {\sum }_{( < {x}_{i}^{p} , {t}_{i}^{p} > , < {x}_{i + 1}^{p} , {t}_{i + 1}^{q } > ) ∈ S} {[ \frac{{w}^{T} ( {x}_{i + 1}^{p} - {x}_{i}^{p} )}{{t}_{i + 1}^{p} - {t}_{i}^{p}} ]}^{2} \qquad ( 8 )$$

Here, x represents a feature vector derived from the sensor data recorded during activities collected using HopkinsPD at a given time. A total of 435 features were computed from the five smartphone-enabled test activities. For example, 126 features were computed from the gait and balance tests each to capture changes in body motion, including the mean, median, standard deviation, range, entropy, and dominant frequency from the tri-axial acceleration time-series. 151 features were computed from the tapping test screen touch events, to quantify attributes such as finger tapping speed (e.g., total number of taps within a given period of time), precision of tapping (e.g., range of tap positions normalized by smartphone screen size), and rhythm and inter-tap interval. Each i, j is a numerical index associated with two distinct timestamps, at times tᵢ and tⱼ, at which activities were conducted. Each p, q represents two distinct patient indices. The vector w is a vector of weights estimated by DSSL. To compute the mPDS on a new patient at a given time t given a recording of their activities at that time and the resulting feature vector x computed from the sensor data collected during these activities, the linear projections w·x are computed. These linear projections are raw and unscaled. To ease interpretability in a clinical setting, the mPDS is scaled between 0 and 100, where values close to 0 reflect low severity while those close to 100 reflect high severity.

The set O is the set of all available pairs of tuples (<xᵢ^{p}>, <xⱼ^{q},tⱼ^{q}>) that are ordered by severity; from the “remote active users” cohort, such pairs are computed automatically based on the activities performed at times right before medication administration and those from the hour after. Severity is assumed to be lower post medication administration. In the second term in Eq. 8, Lₕ is the Huber loss function. This second term in the objective encourages DSSL to estimate a score that satisfies the severity ordering prescribed by the tuples in set O. There were a total of 10,152 such pairs available in the “remote active users” cohort.

The set S, denoted by pairs of tuples (<xᵢ^{p},tᵢ^{p}>, <x^{q}ᵢ₊₁,t^{p}ᵢ₊₁>), are obtained based on tests taken at consecutive times within a few hours of each other but without medication administration during the interim period. The third term in Eq. 8 encourages temporal smoothness for the pairs specified in set S. The coefficients O and S are DSSL regularization parameters and control the relative degree of emphasis on the smoothness between consecutive pairs in the third term of the objective versus maximizing the difference in severity for pairs specified in the second term. These were set using 10-fold cross-validation on the active users cohort.

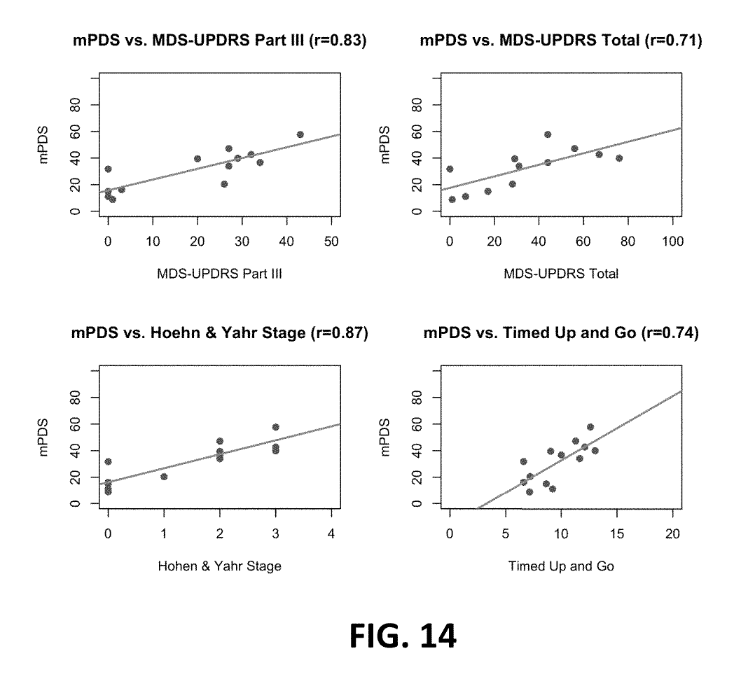

The in-clinic validation cohort was used to validate the mPDS against traditional clinical measures. Individual performance on conventional clinical assessments was compared to the smartphone-derived score of the present invention using correlation plots of mPDS against the MDS-UPDRS part III score, MDS-UPDRS total score, Timed Up and Go Test, and Hoehn & Yahr stage obtained within a 90-minute window of completing the smartphone activities. This 90-minute constraint was chosen to limit PD symptom variability, and to ensure that the smartphone assessments used to calculate the mPDS were performed in the same motor state as the corresponding clinical assessments. In addition to the 90-minute constraint, aberrant measurements were filtered by removing first-time log-ins into the app and smartphone assessments in this cross-sectional analysis deemed outliers by iterative application of Grubb's test for outliers, a standard approach to outlier detection. If at least 10% of sensor-readings (features) on a smartphone assessment for any of the five activities were deemed outliers by Grubb's test (with respect to that user's typical sensor-readings on that activity), that smartphone assessment was excluded from the analysis. The rationale behind excluding first time uses of the application stemmed from an observation that a large proportion of first time uses met the above criteria for aberrant measurements. After filtering these assessments, the pairwise correlations between traditional measures and mPDS was documented and is illustrated in FIG. 14 and described in Table VI, below. Users with incomplete in-person assessments were necessarily excluded. FIG. 14 illustrates graphical views of correlation of mobile Parkinson Disease Score (mPDS) with traditional Parkinson disease rating scales (n=12 for MDS-UPDRS total score, n=13 for others).

TABLE VI
Correlation matrix among mobile Parkinson Disease Score (mPDS) and
conventional assessments (n = 12 for MDS-UPDRS total score,
n = 13 for others).

| | MDS- | Timed Up | MDS- | Hoehn & | |
|---|---|---|---|---|---|
| | UPDRS | and Go | UPDRS | Yahr | |
| | Part III | Time | Total | Stage | mPDS |
| MDS-UPDRS | 1.00 | 0.76 | 0.80 | 0.93 | 0.83 |
| Part III | | | | | |
| Timed Up and Go | 0.76 | 1.00 | 0.84 | 0.88 | 0.74 |
| Time | | | | | |
| MDS-UPDRS | 0.80 | 0.84 | 1.00 | 0.90 | 0.71 |
| Total | | | | | |
| Hoehn & Yahr | 0.93 | 0.88 | 0.90 | 1.00 | 0.87 |
| Stage | | | | | |
| mPDS | 0.83 | 0.74 | 0.71 | 0.87 | 1.00 |

MDS-UPDRS = Movement Disorder Society-Unified Parkinson's Disease Rating Scale;
mPDS = mobile Parkinson Disease Score

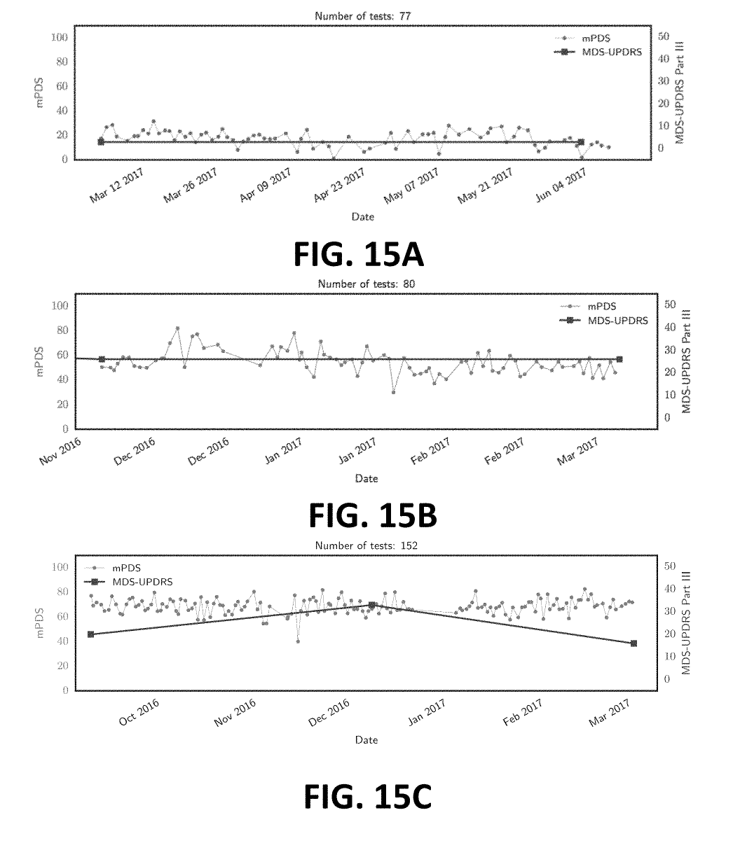

To demonstrate the utility of mPDS in visualizing intraday variability not captured by MDS-UPDRS, mPDS and MDS-UPDRS part III score trajectories were plotted for three participants, as illustrated in FIGS. 15A-15C. FIGS. 15A-15C illustrate graphical views of sample longitudinal assessments of individuals over six months using the mPDS and the MDS-UPDRS Part III motor score. More particularly, FIG. 15A illustrates a graphical view of change over six months in mPDS and MDS-UPDRS part III scores for an individual without Parkinson disease; FIG. 15B illustrates a graphical view of change over six months in mPDS and MDS-UPDRS part III scores for an individual with moderate-severe Parkinson disease (Hoehn and Yahr stages II-III); and FIG. 15C illustrates a graphical view of change over six months in mPDS and MDS-UPDRS part III scores for an individual with severe Parkinson disease (Hoehn and Yahr stage III). The absolute change in mPDS was also calculated for participants in both the in-clinic validation and active user cohorts, defined for each patient as the difference between their maximum and minimum mPDS scores for each day, averaged over all days of that patient's enrollment in the study.

Correlation of change in MDS-UPDRS part III and mPDS between off- and on-medication evaluations was evaluated. A one-tailed Wilcoxon signed-rank test was used to assess the significance of average intraday reduction, the mean difference in mPDS between the off-medication and on-medication states, being greater than 0 (consistent with the expectation that severity is higher in the off state). The mPDS and MDS-UPDRS part III from all patients are displayed with off-medication and on-medication evaluations conducted within the same day. This is provided to assess the extent to which changes in mPDS tracked those in MDS-UPDRS after medication.

On the active users cohort, an analogous Wilcoxon signed-rank test was conducted to assess significance of average intraday reduction in mPDS after dopaminergic therapy. Two examples in this cohort were also examined in more detail. Rather than choosing these individuals at random, they were selected as representative examples in which mPDS correctly ordered the severity states in the majority of paired instances for one patient with a stable long-term trajectory and for another who worsened over six months.

Of the 250 individuals with PD from 12 countries who downloaded the HopkinsPD Android application in the first recruitment phase, 139 were active users. 22 individuals with PD and 17 individuals without PD were additionally recruited to the in-clinic validation cohort (baseline characteristics in Table VII). The 22 individuals with PD completed a total of 51 assessments (22 at baseline, 16 at month 3, and 13 at month 6); the 17 individuals without PD completed 35 assessments (17 at baseline, 11 at month 3, and 7 at month 6).

TABLE VII
Characteristics of the study populations at the time of enrollment.

| | All | | | |
|---|---|---|---|---|
| | HopkinsPD | “Remote active | | |
| | users with | users” with | In-clinic validation | In-clinic validation |
| | Parkinson | Parkinson | cohort with | cohort without |
| | disease | disease | Parkinson disease | Parkinson disease |
| Characteristic | n = 250 | n = 139 | n = 22 | n = 17 |
| Demographics | | | | |
| Age (years) | 57.2 (9.4) | 58.7 (8.6) | 64.6 (11.5) | 54.2 (16.5) |
| Sex (% women) | 38 | 43 | 48 | 71 |
| Race (% white) | 90 | 95 | 95 | 94 |
| Ethnicity (% | 6 | 7 | 0 | 0 |
| Hispanic/Latino) | | | | |
| Education (% | 95 | 94 | 62 | 47 |
| college | | | | |
| graduate) | | | | |
| Using the | 100 | 100 | 91 | 100 |
| interne or email | | | | |
| at home (%) | | | | |
| Clinical | | | | |
| characteristics | | | | |
| Time since | 4.4 (4.9) | 4.3 (4.4) | 7 (4.1) | NA |
| diagnosis (years) | | | | |
| Proportion | 96 | 97 | 90 | NA |
| taking levodopa | | | | |
| (%) | | | | |
| Years taking | 4.4 (4.9) | 4.3 (4.4) | 7 (4.3) | NA |
| Parkinson | | | | |
| disease | | | | |
| medications | | | | |
| (years) | | | | |
| MDS-UPDRS, | NA | NA | 26.9 (11.2) | 1.2 (1.7) |
| part III score | | | | |
| MDS-UPDRS, | NA | NA | 55.0 (26.5) | 4.6 (4.6) |
| total (I + II + III) | | | | |
| score | | | | |
| Timed Up and | NA | NA | 11.2 (3.3) | 8.1 (1.3) |
| Go Test | | | | |
| (seconds) | | | | |
| Hoehn & Yahr | NA | NA | 2.1 (0.7) | 0.0 (0.0) |

Mean (standard deviation) pairs listed except where indicated.
NA = Not available
MDS-UPDRS = Movement Disorder Society-Unified Parkinson's Disease Rating Scale

In the active users cohort, individuals performed an average of 98 complete sets of smartphone tasks over six months. Similarly, patients with PD in the in-clinic validation cohort performed an average of 115 complete sets of smartphone tasks over the same timeframe.

Eight features from the finger tapping activity, three features from the balance activity, three features from the gait activity, and one feature from the voice activity contributed most toward generating mPDS values. A detailed description of these 15 features with highest weight contribution to the mPDS is provided in Table VIII. The relative weighting of the features from each activity in the model's determination of a patient's underlying severity state was as follows: gait (35.4%), finger tapping (23.6%), balance (21.5%), voice (17.4%), and reaction time (2.1%).

TABLE VIII
The top fifteen features determined by ranking mPDS' absolute feature weights

| Smartphone test | |
|---|---|
| protocol | Feature description |
| Finger tapping | Mean vertical tapping position scaled according to smartphone screen size |
| Balance | Mean acceleration in the direction of motion when the individual is walking |
| Gait | Entropy of the acceleration in the direction of motion when the individual is walking |
| Finger tapping | Mean vertical tapping position on the left button scaled according to smartphone |
| | screen size |
| Finger tapping | Mean square energy of the vertical tapping position scaled according to |
| | smartphone screen size |
| Balance | Mean acceleration in the direction of the gravitational acceleration vector |
| Gait | Entropy of the acceleration in the side direction (perpendicular to the walking |
| | direction) |
| Finger tapping | Mean horizontal tapping position on the left button scaled according to smartphone |
| | screen size |
| Finger tapping | Mean squared energy of the vertical tapping position on the right button scaled |
| | according to smartphone screen size |
| Gait | Entropy of acceleration in the direction of the gravitational acceleration vector |
| Finger tapping | Mean horizontal tapping position on the left button scaled according to smartphone |
| | screen size |
| Finger tapping | Median vertical tapping position on the left button scaled according to smartphone |
| | screen size |
| Finger tapping | Mean squared energy of the vertical tapping position on the left button scaled |
| | according to smartphone screen size |
| Balance | Entropy of acceleration in the inclination direction in the spherical coordinate |
| | system |
| Voice | Mean voice amplitude over all 0.5 second frames with voiced signal |

A total of 13 complete smartphone task (mPDS) and in-clinic assessment pairs among 9 individuals met the criteria for analysis and were used in the cross-sectional analysis between mPDS and traditional outcome measures. Among the excluded assessment pairs, there was 1 incomplete in-clinic assessment (for MDS-UPDRS total score), 12 first-time smartphone assessments, and 5 additional smartphone assessments that met the exclusionary criteria by iterative application of Grubb's test; of these 12 first-time smartphone assessments, 8 met the criteria for exclusion by Grubb's test. In fact, at least 10% of sensor readings on one or more smartphone activities were >3 standard deviations from that participant's typical readings (for that activity) on 12/13 assessments excluded by Grubb's test.

FIG. 14 illustrates correlations between mPDS and traditional measures across all eligible mPDS scores computed within 90 minutes of a clinical assessment. The mPDS was well correlated with the motor (part III) portion of the MDS-UPDRS (r=0.83), the Hoehn & Yahr stage (r=0.87), the Timed Up and Go Test (r=0.74), and the total MDS-UPDRS (r=0.71). The correlations between mPDS and other conventional scores are similar to those that exist between these well-established rating scales (Table VI).

FIGS. 15A, 15B, and 15C show three representative individuals tracked over six months by the mPDS and who performed three MDS-UPDRS part III assessments at baseline, Month 3, and Month 6. Both the mPDS and MDS-UPDRS part III motor scores show low scores and low variability for the individual without PD (FIG. 15A). In FIG. 15B, both scales trace moderate, stable severity trajectories for an individual with Hoehn and Yahr stage II PD; however, the mPDS captures daily variation, particularly two months after this patient's baseline in-clinic assessment, which the MDS-UPDRS could not practically detect. FIG. 15C represents a patient with Hoehn and Yahr stage III PD. The mPDS reflects a relatively high severity score for this participant and captured significant intraday variability. However, the overall mPDS trajectory remained stable over the 6-month observational period, indicating relative stability in disease severity. In contrast, the patient's 3 MDS-UPDRS part III assessments demonstrate significant variation in score over the observational period, making statements regarding the patient's disease trajectory (with only 3 points) difficult, and illustrating the potential for false or missed signals with episodic outcome measures.

For the in-clinic validation cohort, the average absolute change in mPDS was 14.9 (S.D. 8.0). Average intraday reduction in mPDS due to dopaminergic therapy was significant (against a null hypothesis of no change, test statistic of 15, n=5, p=0.031, one-tailed Wilcoxon signed rank test), leading us to conclude that patients in this cohort saw a reduction in mPDS (as expected) after medication.

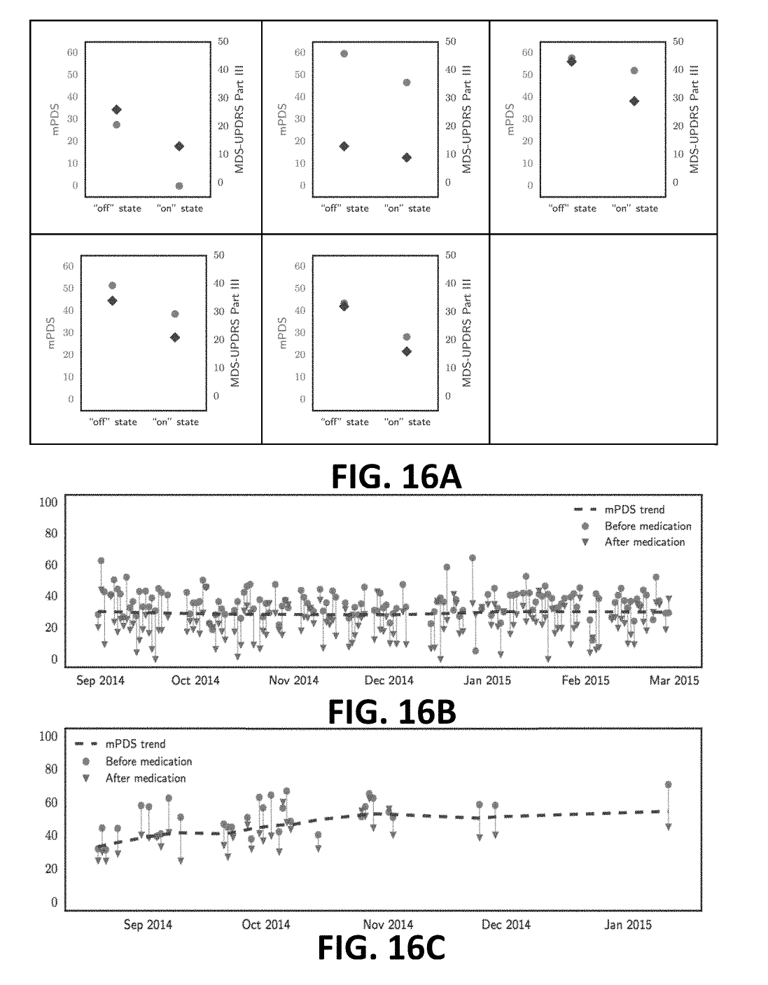

FIGS. 16A-16C illustrate graphical views of evaluations of change in mPDS in response to dopaminergic therapy. In FIG. 16A, red points map to mPDS scores; blue points map to MDS-UPDRS part III motor scores. In FIGS. 16A and 16B, (1) circle points label scores computed before first daily levodopa dose, (2) triangles label those computed within 90 minutes after this first dose, (3) connecting lines yield estimated score change pre- and post-levodopa administration, (4) dashed line shows a moving average of mPDS over duration of observation.

FIG. 16A details the off- and on-medication changes in mPDS for 5 patients who, in addition to completing three MDS-UPDRS assessments at baseline, month 3, and month 6, also completed the optional off-medication and on-medication mPDS assessments at month 6. As shown, for each patient, mPDS and MDS-UPDRS part III decrease after medication, tracking one another approximately in parallel.

In the active users cohort, average intraday reduction in mPDS between the off- and on-medication states was 4.6 (S.D. 5.4); this was also significant (against a null hypothesis of no change, test statistic of 7086, n=139, p=2.00×10⁻⁷, one-tailed Wilcoxon signed rank test). Overall, the active users saw large changes in mPDS on the same day before and after dopaminergic therapy; the average patient experienced an absolute change in mPDS of 10.0 (standard deviation 13.4) between pre- and post-medication states.

FIGS. 16B and 16C demonstrate these intraday fluctuations in two patients' mPDS trajectories. Both figures illustrate that in the majority of paired off- and on-medication evaluations, mPDS predicted a lower motor symptom severity (lower mPDS) in the on-medication state.

The mPDS provides a rapid, remote measure of PD manifestations that can be assessed on widely available smartphones. The mPDS correlates with classical clinical measures, detects substantial intraday variability, and changes significantly in response to known, effective treatments. That the high correlations between mPDS and traditional measures are similar to those that exist between these well-established rating scales further validates its use (Table VII). Given the increasing ubiquity of smartphones and the need for inexpensive, objective measures, an automated, mobile measure of PD could be of substantial value toward assessing the efficacy of existing and new therapies for PD in the short-term and improving clinical care in the long-term.

The mPDS could be a powerful complement to traditional measures. First, the mPDS can be assessed almost anywhere an individual with PD is located. Such “real-world” data is increasingly sought by regulators, such as the U.S. Food and Drug Administration. Second, the mPDS can be calculated at almost any time with high frequency, allowing detection of substantial intraday variability. This is not possible using traditional episodic clinical assessments like the MDS-UPDRS, as these would need to be completed by each patient in clinic, multiple times per day. Third, the score is objective and not subject to the availability and variability of raters, improving both its clinical utility and its potential use as an outcome measure in clinical trials. Fourth, by gathering input from 435 unique features, the mPDS captures and weighs key aspects of the disease that may be over- or underrepresented in traditional measures. For example, features from the gait and balance tasks were responsible for 56.9% of mPDS score calculation, yet the motor portion of the MDS-UPDRS devotes only three questions to gait and balance. Last, through its relative weighting, the mPDS gives greater priority to more “important” symptoms, while traditional measures give equal weight to all features, even those that do not contribute meaningfully to overall disease severity. Thus, the mPDS provides guidance on the utility of each of the five smartphone tests toward assessing symptom severity.

This study has several design, scope, and technical limitations. While participants were drawn from 12 countries, participation was limited to those with access to the necessary technology. This population is a direct reflection of the digital divide and may limit its generalizability to the broader population affected by PD. In addition, as demonstrated by other smartphone research studies′, use of HopkinsPD declined rapidly. Nearly two thirds of participants discontinued use of the application after one month. The HopkinsPD application currently does not provide feedback to participants on their own scores or on how they are performing relative to others like them; addressing this limitation is likely to lead to stronger long-term adoption.

Cardinal features of PD, such as rest tremor, were not measured with this smartphone application. Common non-motor features, such as depression, anxiety, sleep difficulties, and cognitive impairment, are not currently captured by mPDS. The inclusion of such information via modalities such as passive monitoring of GPS, language use in texts, cognitive tasks, and assessment of vital signs (e.g., heart rate) all provide additional opportunities for developing a more comprehensive assessment of how PD affects individuals.

From a technical standpoint, the study was conducted on multiple types of Android smartphones. These smartphones may have different sensors (e.g., accelerometers) and different versions of the operating system that may have even changed over the course of the study. All of these limitations would have introduced more “noise” into the data. This study also relied on self-reported data (e.g., of medication administration), which may not have always been accurate. However, the out-of-clinic changes in response to levodopa appear to be mirrored by those performed in clinic.

Last, the study is limited by the small number of paired in-person assessments with smartphone tasks. This small number was driven by a number of factors. First, while a set of smartphone tasks was performed within 90 minutes of each in-person clinician-performed assessment, problems with time stamping and flagging the in-clinic performed smartphone tasks limited the ability to pair many of these assessments together. In ongoing validation of the mPDS, it will be essential to more definitively timestamp and mark this data. Second, of those assessments that were completed within 90 minutes of each other, more than half were excluded from the analysis; a majority of these exclusions was related to first-time use of the application and artificially elevated scores, likely reflecting participant inexperience with the tasks, rather than true motor performance. Orientation to the tasks prior to the first attempt may be useful in the future to improve the validity of all assessments. Still, despite these limitations and the small sample numbers, correlations remain excellent. Validation on a larger sample size will be useful to further validate the score's utility.

In conjunction with the present invention, visualizations of the patient's scoring can be provided to a health care professional. This information can be provided as averages of all of the assessments and/or scores for specific assessments. Trends over time and areas of concern can be highlighted with graphs, heatmaps, or other suitable visualizations. On the patient side, the present invention prompts the patient to participate in the assessments. Alerts can be sent to the patient when assessments need to be completed and alerts can be sent to healthcare providers to prompt patients to participate in the assessments. These alerts can be pushed directly to the patient or healthcare providers device whether the device is on or off. Further, automatic triggering of certain assessments can also be initiated, such as gait assessment, tapping assessment, or speech assessment, with the smartphone application running in the background of the patient's phone. Each of these activities is executed by the patient in conjunction with the smartphone, likely several times per day, and this more continuous or spontaneously initiated assessment can obtain additional data points when the patient is acting naturally throughout her day.

The steps and analysis of the present invention can be carried out using a smartphone, a tablet, internet or cellular enabled device, computer, non-transitory computer readable medium, or alternately a computing device or non-transitory computer readable medium incorporated into the imaging device. Indeed, any suitable method of calculation known to or conceivable by one of skill in the art could be used. It should also be noted that while specific equations are detailed herein, variations on these equations can also be derived, and this application includes any such equation known to or conceivable by one of skill in the art. A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computing device designed specifically for this purpose. The computing device can be unique to the present invention and designed specifically to carry out the method of the present invention.

The many features and advantages of the invention are apparent from the detailed specification, and thus, it is intended by the appended claims to cover all such features and advantages of the invention, which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method for Parkinson's disease (PD) monitoring and intervention for a patient comprising:
collecting passive and active data related to the patient using a smartphone with an application, wherein active data includes the application prompting tests of gait, voice, screen tapping, and posture and passive data includes information collected by the application via features of the smartphone in the background of operation of the smartphone;
analyzing the passive and active data;
transforming the passive and active data into visual representations of the data for a health care provider; and
providing updates and reminders to the patient.

2. The method of claim 1 wherein passive data further comprises data from accelerometers, inertial sensors, GPS, WiFi, and phone usage.

3. The method of claim 1 further comprising prompting the patient to perform active data testing.

4. The method of claim 1 further comprising prompting the patient to take medicine.

5. The method of claim 1 further comprising providing a smartphone for collection of the active and passive data.

6. The method of claim 1 further comprising transmitting the visual representation of the data to the healthcare provider.

7. The method of claim 1 further comprising prompting the patient to participate in assessments of gait, voice, screen tapping, and posture.

8. The method of claim 1 further comprising transmitting advice from the health care provider to the patient.

9. The method of claim 1 further comprising adjusting patient medication dosage based on the passive and active data.

10. The method of claim 1 further comprising analyzing the passive and active data with a rank-based machine learning algorithm.

11. A system for Parkinson's disease (PD) monitoring and intervention for a patient comprising:
a smart device comprising sensors;
a processor configured to execute a non-transitory computer readable medium, wherein the non-transitory computer readable medium is programmed for:
collecting passive and active data related to the patient using a smartphone with an application, wherein active data includes the application prompting tests of gait, voice, screen tapping, and posture and passive data includes information collected by the application via features of the smartphone in the background of operation of the smartphone;
analyzing the passive and active data;
transforming the passive and active data into visual representations of the data for a health care provider; and
providing updates and reminders to the patient.

12. The system of claim 11 wherein the sensors comprise accelerometers, inertial sensors, GPS, WiFi, and phone usage.

13. The system of claim 11 further comprising prompting the patient to perform active data testing.

14. The system of claim 11 further comprising prompting the patient to take medicine.

15. The system of claim 11 further comprising providing a smartphone for collection of the active and passive data.

16. The system of claim 11 further comprising transmitting the visual representation of the data to the healthcare provider.

17. The system of claim 11 further comprising prompting the patient to participate in assessments of gait, voice, screen tapping, and posture.

18. The system of claim 11 further comprising transmitting advice from the health care provider to the patient.

19. The system of claim 11 further comprising adjusting patient medication dosage based on the passive and active data.

20. The system of claim 11 further comprising analyzing the passive and active data with a rank-based machine learning algorithm.
